(19) United States
(12) Patent Application Publication
(10) Pub. No.: US 2007/0005122 A1
(43) Pub. Date: Jan. 4, 2007

(54) Treatment instrument installation member, stent delivery catheter and conduit system

(75) Inventor: Yoshimitsu Inoue, Tokyo (JP)

Correspondence Address: Thomas Spinelli;Scully, Scott, Murphy & Presser, 400 Garden City Plaza, Garden City, NY, 11530, US

(73) Assignee: Olympus Corporation, Tokyo (JP)

(21) Appl. No.: 11/515,591

(22) Filed: Sep. 5, 2006

Related U.S. Application Data
(63) Continuation of application No. PCT/JP05/17196, filed on Sep. 16, 2005.

(30) Foreign Application Priority Data
Sep. 24, 2004 (JP) P2004-277650

(51) Int. Cl. A61F 2/06

(52) U.S. Cl. 623/1.11

(57) ABSTRACT

A stent delivery catheter, which is a treatment instrument, has an elongated guide catheter which is to be inserted into an endoscope and the human body. The stent is provided around a distal end of a guide catheter. Furthermore, a stent installation member which can cover a flap provided on a proximal end of the stent is provided thereon. The distal end of a stent installation member is inserted so that it reaches a main conduit passing through a first conduit and a bifurcation provided inside of the endoscope. The flap provided on the proximal side of the stent and the proximal end of the stent is inserted into a main conduit passing through the stent installation member. The stent is prevented from becoming stuck or the like when the treatment instrument is inserted into a conduit system of the endoscope.

### BACKGROUND OF THE INVENTION

1. Field of the Invention

The present invention relates to a treatment instrument installation member, a stent delivery catheter provided with the treatment instrument installation member and a conduit system. This application is a continuation application based on a PCT Patent Application No. PCT/JP2005/017196, filed on Sep. 16, 2005, whose priority is claimed on Japanese Patent Application No. 2004-277650 filed on Sep. 24, 2004. The contents of both the PCT Application and the Japanese Application are incorporated herein by reference.

2. Description of the Related Art

When stenosis develops at the bile duct or the like inside the human body, it is well known to secure a passage of the bile duct or the like by inserting an elongated tubular stent into the bile duct or the like and further inserting through the stent inside of the stenotic area. At the time of inserting the stent, the stent is provided on an elongated guide member, and a guide member is inserted through a conduit of an endoscope into the inside of the bile duct or the like from the tip of the endoscope and then the stent is pushed out of the guide member. The stent is provided with flaps and by opening these flaps the stent is prevented from being dislodged from the stenosis area.

Conventionally, at the time of inserting the stent, a stent installation member to guide the stent into the conduit of endoscope has been used. The stent installation member has substantially the same inner diameter as the opening of the insertion slot of the conduit and the stent installation member is made so that the stent provided inside of the stent installation member is guided into the conduit if the stent installation member is pressed against the surroundings of the insertion slot of the conduit.

Because the flap provided on the proximal side of the stent is made to open towards the distal end of the stent, it is possible for the flap on the proximal side to bent due to interference with the insertion slot of the conduit at the time of inserting the stent. Because of this, an invention such as Japanese Unexamined Patent Application, First Publication No. H11-76412 is disclosed that enables the tip of the stent installation member to be inserted into the insertion slot of the conduit. The stent installation member has a length to completely cover the flap provided on the proximal side of the stent and therefore the flap on the proximal side is kept covered by the stent installation member and inserted into the insertion slot of the conduit.

### SUMMARY OF THE INVENTION

According to a first embodiment of the present invention, a treatment instrument installation member is provided which is used when the first treatment instrument is inserted into the first conduit of the conduit system that includes the first conduit provided with an inner diameter through which the first treatment instrument can be inserted and the second conduit provided with an inner diameter through which the second treatment instrument can be inserted, and the main conduit provided with an inner diameter through which the first and the second treatment instrument can be inserted and a connection part that connects the first conduit, second conduit and the main conduit together. The treatment instrument insertion member is comprised of a main body which has an outer diameter that can be inserted into the first conduit and a distal end part located on the distal end of the main body that has a blocking part which blocks at least a part of the opening through which the second conduit communicates to the connection part.

Preferably, the main body of the above mentioned treatment instrument installation member is a tubular member which has an outer diameter smaller than the inner diameter of the first conduit and that of the connection part and is provided with an inner diameter larger than that of the outer diameter of the first treatment instrument, and the distal end portion is exposed from the conduit system at the insertion slot which is provided on a side where the operating area for the conduit system is provided while the distal end part blocking at least a part of the opening communicating with the second conduit at the connection part.

Preferably, the main body of the above mentioned treatment instrument installation member is provided with a regulative part on its proximal end which regulates the installation amount of the main body into the first conduit.

Preferably, a stent delivery catheter is provided that is used with an endoscope including the first conduit provided with an inner diameter through which the first treatment instrument can be inserted, the second conduit provided with an inner diameter through which the second treatment instrument can be inserted, the main conduit provided with an inner diameter through which the first and the second treatment instrument can be inserted and a connection part that connects the first conduit, second conduit and the main conduit together. The stent delivery catheter comprises a guide member inserted into the first conduit and the main conduit from the insertion slot for the first treatment instrument provided at the operation area of the endoscope, a stent which is movable forward and backward which is provided on the guide member, a pushing member which is movable forward and backward which is provided on the guide member to push the stent out of the guide member to be placed in the human body, and a stent installation member which corresponds the above-mentioned treatment instrument installation member which is movable forward and backward which is provided on the pushing member and on the stent.

Preferably, the stent of the above mentioned stent delivery catheter is provided with flaps which regulate the movement of the stent, and the stent delivery catheter is provided with a protective case which covers the stent with its flaps open and with the stent provided on the guide member.

Preferably, an endoscope treatment system provided with the endoscope and the stent delivery catheter is provided.

Preferably, a conduit system is provided which comprises the first conduit which is provided with an inner diameter through which the treatment instrument can be inserted, the second conduit which is provided with an inner diameter through which the second treatment instrument can be inserted, the main conduit which is provided with an inner diameter through which the first and the second treatment instrument can be inserted, a connection part which connects the first conduit, second conduit and the main conduit together, and a regulative member which is provided at the connection part and prevents the first treatment instrument inserted from the first conduit from proceeding into the second conduit and at the same time enables the second treatment instrument inserted from the second conduit to proceed into the main conduit.

Preferably, a stent installation method is provided which is utilized when the stent, which has flaps on its distal end and proximal end each to be placed in the human body, is inserted from the insertion slot provided on the divaricated conduit divaricated at the bifurcation from the main conduit of the endoscope which has an opening on its distal end. The stent installation method includes a stent installation member insertion process and stent insertion process. The stent installation member insertion process is to insert the stent installation member, through which the stent can be inserted and which is longer than the route from the insertion slot to the bifurcation of the main conduit, from the insertion slot of the divaricated conduit and to enable the distal end of the stent installation member to pass through the bifurcation and to reach the main conduit after placing at least the flap of the stent provided on the proximal end of the stent inside of the stent installation member. The stent insertion process is to make at least the flap of the stent provided on its proximal end pass through the bifurcation by passing through the stent installation member after the stent installation member insertion process.

### BRIEF DESCRIPTION OF THE DRAWINGS

FIG. 1 is an overall view of an endoscope treatment system including a stent delivery catheter according to a first embodiment of the present invention.

FIG. 2 is a cross section of a partially enlarged side view of the endoscope treatment system of FIG. 1.

FIG. 3 is an overall view showing a structure of the stent delivery catheter.

FIG. 4 is a view explaining a usage of the stent delivery catheter.

FIG. 5 is a cross section of a partially enlarged side view of the endoscope treatment system of FIG. 1, showing a stent inserted into a main conduit.

FIG. 6 is a view showing the stent inserted into a living body.

FIG. 7 is a view showing the stent.

FIG. 8 is a view showing the stent delivery catheter and its protective case.

FIG. 9 is a side view of the protective case.

FIG. 10 is a side view of the stent installation member according to a second embodiment of the present invention.

FIG. 11 is a view showing the installation of the stent.

FIG. 12 is a side view showing the stent installation member inserted into the endoscope.

FIG. 13 is a side view of the stent installation member.

FIG. 14 is a side view showing the stent installation member inserted into the endoscope.

FIG. 15 is a side view of the stent installation member according to a third embodiment of the present invention.

FIG. 16 is a side view showing the stent installation member inserted into the endoscope according to a fourth embodiment of the present invention.

FIG. 17 is a view of a regulative member seen from the conduit B-B toward the direction of the arrow in FIG. 16.

### DETAILED DESCRIPTION OF THE INVENTION

The best mode for carrying out the invention of the present application is explained by referring to the drawings.

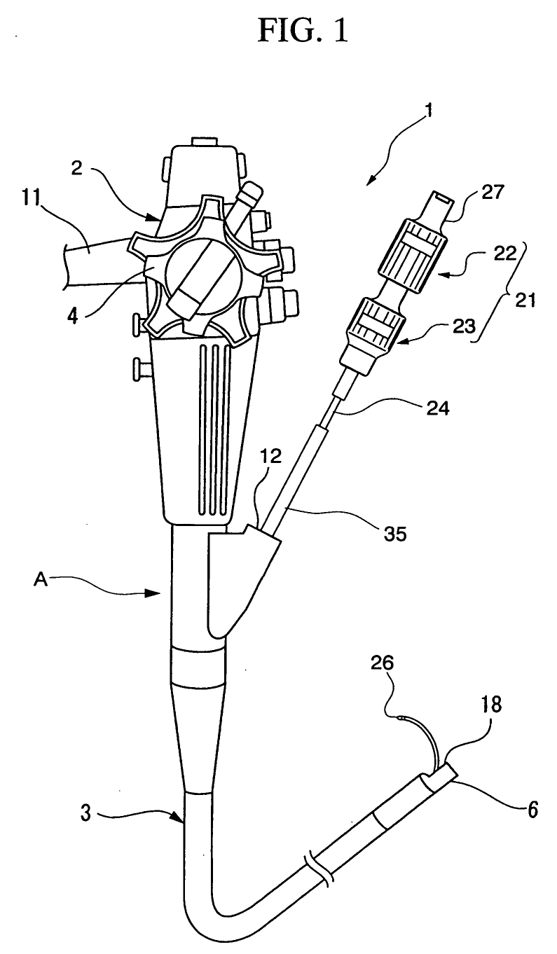

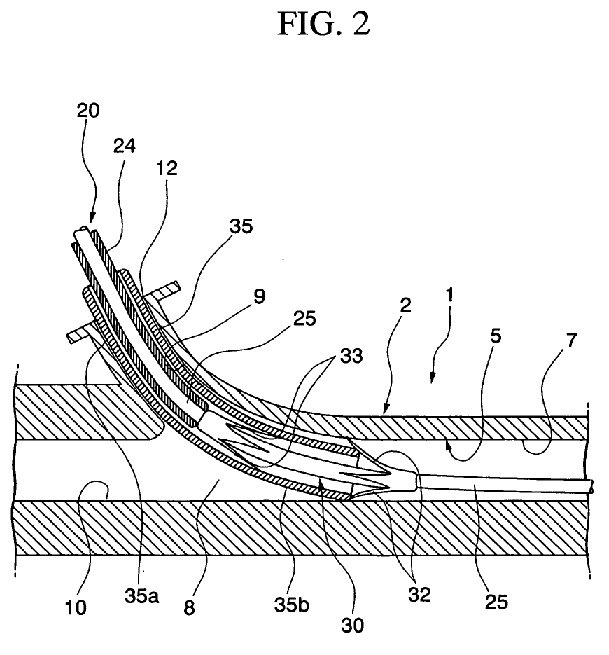

FIG. 1 is a view showing a stent delivery catheter according to a first embodiment of the present invention inserted through an endoscope and FIG. 2 is an enlarged view of the cross section of part A of FIG. 1.

As shown in FIGS. 1 and 2, an endoscope 1 has an endoscope operation part 2 that an operator operates and, on the tip of the endoscope operation part 2, an endoscope insertion part 3 is provided to be inserted into the human body. The endoscope insertion part 3 is flexible and can be bent by the operation of a knob 4 of the endoscope operation part 2. A treatment instrument insertion channel 5, which is a conduit system, is provided inside of the endoscope 1. A distal end 6 of the treatment instrument insertion channel 5 has an opening on the side of the distal end 6 of the endoscope insertion part 3 and communicates with the endoscope operation part 2 through the endoscope insertion part 3.

The treatment instrument insertion channel 5 comprises a main conduit 7 which communicates with the distal end 6 of the endoscope insertion part 3 and a first conduit 9, and is divaricated to a second conduit 10 at a bifurcation 8 which is a connection part. The second conduit 10 extends along the axis of the main conduit 7 and communicates with a liquid supply conduit and air supply conduit which are both not shown, and can supply normal saline as a second treatment instrument. The liquid supply conduit or the air supply conduit are communicate with a liquid supply apparatus or an air supply apparatus provided outside via a universal cord 11 which extends from the side of the endoscope operation part 2 as shown in FIG. 1. The first conduit 9 is extended to an insertion slot 12 provided on the side of the endoscope operation part 2. A stent delivery catheter 20 is inserted as the first treatment instrument into the insertion slot 12.

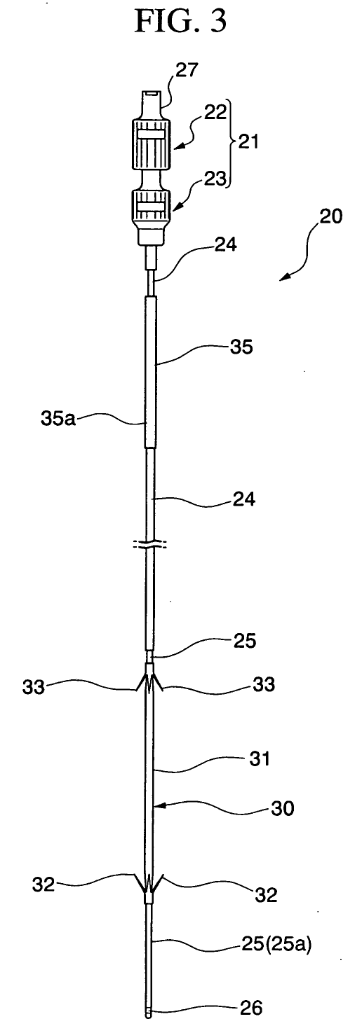

As shown in FIGS. 1 and 3, the stent delivery catheter 20 has an operation part 21 with which an operator operates. An operation part 21 comprises a mouth ring 22 for a guide catheter and a mouth ring 23 for a pushing member which is removably provided on the mouth ring 22. The mouth ring 23 is provided with a pushing member 24 on the distal end of the mouth ring 23. The pushing member 24 comprises a flexible elongated tubular member. A guide catheter 25, which is a guide member, is movably inserted forward and backward through the pushing member 24. The guide catheter 25 comprises a flexible member and is provided with lumens and a marker 26 for X-ray imaging which is buried on the distal end of the guide catheter 25. The lumens, which is passing through inside of the mouth ring 23, and communicate with a connection part 27 provided on the proximal end of the mouth ring 22. The connection part 27 comprises a luer-lock or the like and can communicate with syringes or the like which are not shown in figures.

The guide catheter 25 is longer than the pushing member 24 and the distal end of the guide catheter 25 is exposed from the distal end of the pushing member 24 when the mouth ring 22 and the mouth ring 23 are connected. A stent 30 is movably provided forward and backward around the exposing distal end 25a.

The stent 30 is provided with a tubular main stent body 31. The inner diameter of a main stent body 31 is substantially the same as the outer diameter of the guide catheter 25. The outer diameter of the main stent body 31 is smaller than the inner diameters of those of the main conduit 7 or the bifurcation 8 or the first conduit 9. A flap 32 is provided on the distal end of the stent 30. Four flaps 32 are provided on the circumference of the stent main body 31 and each of the flaps has a 90-degree interval with adjacent flaps, and when a pressure is not applied they are made to open toward the proximal end of the stent 30 by lifting the tip of the flap which is closer to the proximal end. And when a prescribed pressure is applied they are capable of changing their shapes to close themselves. Each of the four flaps 32 is made so as to gradually reduce its width from the proximal end, which is connected to the main stent body 31, to its distal end. Four flaps 33 are provided on the proximal end of the stent 30. The flaps 33 have the same structure as the flaps 32 except that when a pressure is not applied they are made to open toward the distal end of the stent 30 by lifting the four tips of the flap closer to the distal end.

Further, a stent installation member 35 is movably provided forward and backward around the pushing member 24. The stent installation member 35 comprises a tubular main body 35a and the inner diameter of the tubular main body 35a is larger than the outer diameter of the pushing member 24. The inner diameter of the stent installation member 35 is larger than the outer diameter of the stent 30 in a state where both the flaps 32 and 33 are closed. The outer diameter of the stent installation member 35 is smaller than the inner diameter of the first conduit 9 which is located closer to the endoscope 1 and the inner diameter of the bifurcation 8. As explained in detail later, the length of the stent installation member 35 is longer than the total length of the first conduit 9 and is shorter than the pushing member 24 and more preferably, it is approximately 90 mm.

The stent installation member 35 as described above is made of fluorine resin (including PTFE, PFT, FEP or the like) or polyethylene resin or the like. The reason for this is because the stent installation member 35 should be thin, rigid and smooth enough to enable the insertion of the stent installation member 35 into the first conduit 9. And in the stent installation member 35, at least a distal end 35b thereof may be made to be deformable. A structure which enables the distal end 35b of the stent installation member 35 to be deformable is achieved by making, for example, the whole stent installation member 35 or the distal end 35b of the stent installation member 35 with a flexible material or to make the distal end 35b of the stent installation member 35 thinner than other parts or to provide slits on the distal end 35b of the stent installation member 35 along the longitudinal direction or the like. This kind of distal end 35b of the stent installation member 35 acts as a blocking member by reducing the area through which the stent 30 passes at the bifurcation 8.

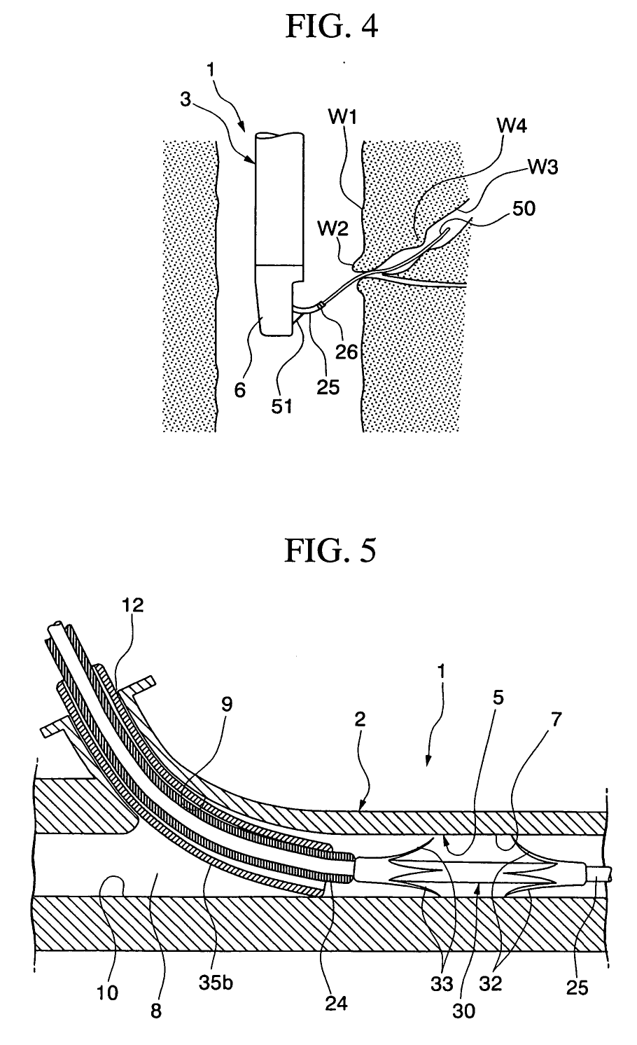

Next, the operation of this embodiment of present invention is explained. The explanation is made referring to an example shown in FIG. 4 which shows a case where the stent 30 is inserted into a bile duct W3 from a duodenal papilla W2 of the duodenum W1 of the patient. In FIG. 4, a guide wire 50 is inserted into the bile duct W3. The guide wire 50 is pre-inserted from the duodenal papilla W2 by using an angiographic tube or the like which is not shown in figures. And when the angiographic tube is pulled out, the distal end of the guide wire 50 remains inside of the bile duct W3 and at the same time the proximal end of the guide wire 50 is pulled outside the body passing through the treatment instrument insertion channel 5 of the endoscope 1.

In the case of inserting the stent 30 as shown in FIG. 3, the mouth ring 22 and the mouth ring 23 are connected first and then the pushing member 24 and the guide catheter 25 are connected via the operation part 21. And then the stent installation member 35 is slid toward the distal end of the guide catheter 25 and the flap 33 provided on the proximal end of the stent 30 is covered with the stent installation member 35.

Next, the proximal end of the guide wire 50 is inserted into the lumen from the distal end of the guide catheter 25 and pulled out from the connection part 27 of the mouth ring 22 of the operation part 21 and then the guide catheter 25 is inserted into the first conduit 9 from the insertion slot 12 of the endoscope 1. Because the outer diameter of the guide catheter 25 is much smaller than the inner diameter of the first conduit 9 and the bifurcation 8, the guide catheter 25 is bent at the bifurcation 8 and led along the main conduit 7 and directed toward the distal end of the main conduit 7. As the guide catheter 25 is directed, the stent 30 and the stent installation member 35 which is provided around the proximal end of the stent 30 are also inserted into the first conduit 9. (Stent installation member insertion process)

The stent 30 is inserted into the first conduit 9 while the flap 32 provided on the distall end of the stent 30 is closed. And the stent 30 is bent along the guide catheter 25 at the bifurcation 8 when the stent 30 proceeds from the first conduit 9 into the main conduit 7. Because the apparent inner diameter is increased at the bifurcation 8, each flap 32 opens temporarily at the bifurcation 8 and closes again when the stent is inserted into the main conduit 7. Because the flaps 32 are made to open by lifting the four tips of the flap closer to the proximal end of the stent 30, the flaps 32 are inserted into the main conduit 7 without being bent as shown in FIG. 2.

The stent installation member 35, which is inserted into the bifurcation 8 following the distal end of the stent 30, is inserted into the main conduit 7 with the distal end of the stent installation member 35 being bent at the bifurcation 8. However, the stent installation member 35, which has substantially the same outer diameter as the inner diameter of the first conduit 9 and has rigidity as a whole, stops as if it fits into the first conduit 9 and the main conduit 7. In this state, by further inserting the guide catheter 25 into the treatment instrument insertion channel 5, the stent installation member 35 remains stationary and the stent 30 moves forward as it is pushed by the pushing member 24. As a result, as shown in FIG. 5, the proximal end of the stent 30 is pushed out of the distal end of the stent installation member 35. Because the flaps 33 are pushed out of the stent installation member 35 after passing through the bifurcation 8, which has an increased inner diameter, even if the flap 33 opens inside of the main conduit 7, the flap 33 is not bent.

And if the guide catheter 25 reaches the distal end of the endoscope 1, as shown in FIG. 4, the position of the distal end of the guide catheter 25 is adjusted to the position of the duodenal papilla W2 by raising a support pillar 51 of the distal end 6 of the endoscope 1. When the positioning is completed, the guide catheter 25 is further pushed and its distal end is inserted inside of the bile duct W3. (Stent installation process)

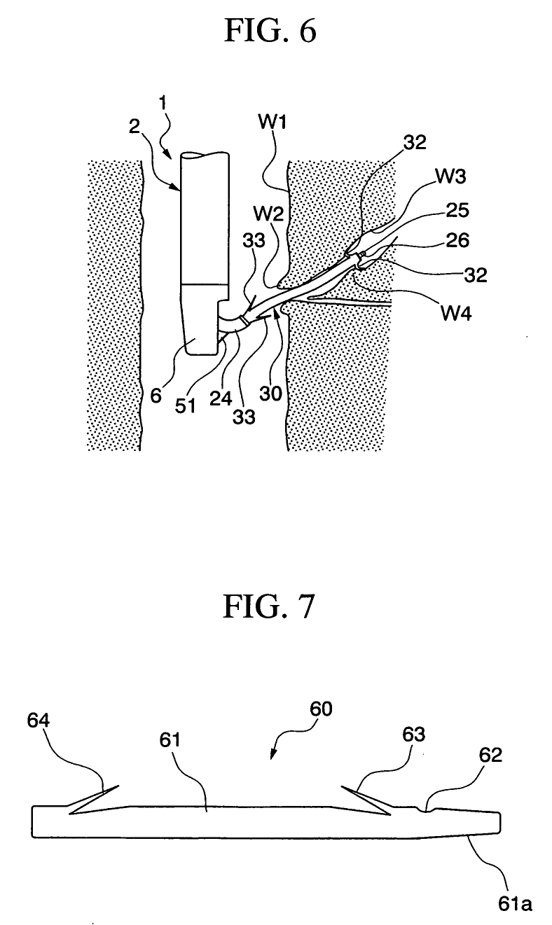

When the marker 26 is inserted so much that it passes over a stenosis W4, the guide catheter 25 is fixed and the mouth rings 22, 23 of the operation part 21 are separated each other and the pushing member 24 is moved forward along the guide catheter 25. When the pushing member 24 moves forward, the distal end of the pushing member 24 pushes the stent 30 toward the distal end of the guide catheter 25. Because of this, as shown in FIG. 6, the distal end of the stent 30 is passed through the duodenal papilla W2 and inserted into the bile duct W3 and is located in an area deeper than the stenosis W4. As the flaps 32 are open toward the proximal end of the stent at this time, they are closed when they pass through the duodenal papilla W2, however, they open again in the area deeper than the stenosis W4 inside of the bile duct W3. The flaps 33 open toward the distal end of the stent 30 inside of the duodenum W1. And when the guide catheter 25 is pulled out in a state that the pushing member 24 is fixed relative to the endoscope 1, then the stent 30 is installed. As a result, the bile duct W3 and the duodenum W1 communicate through a through-hole provided inside of the stent main body 31. A movement of the stent 30 toward the bile duct W3 is regulated by the flap 33 and the movement of the stent 30 toward the duodenum W1 is regulated by the flap 32.

According to this embodiment of the present invention, since the stent installation member 35 is inserted into the treatment instrument insertion channel 5 and the distal end of the stent installation member 35 reaches beyond the bifurcation 8 to the main conduit 7, it is possible to reduce the inner diameter of the space where the stent 30 passes through in the vicinity of the bifurcation 8 and the flaps 33 of the stent 30 are prevented from being open in the bifurcation 8. If the stent installation member 35 is not provided, when the stent proceeds from the bifurcation 8 into the main conduit 7, the flaps 33 which open toward the distal end of the stent 30 tend to be bent because the inner diameter is reduced, however, in this embodiment of the present invention this problem is prevented. Therefore, during the insertion of the stent 30, the bent flap 33 is prevented from getting stuck by the support pillar 51. Furthermore, when the stent 30 is placed in the human body, the stent 30 is not easily dislodged because of the correct functions of the flaps 32 and 33. Because the stent installation member 35 has such a rigidity to be inserted into the first conduit 9 and the distal end of the stent installation member 35 is made to change its shape along the bifurcation 8 and the main conduit 7, deformation or the like of the flaps 33 are prevented by simply inserting the stent installation member 35 into the endoscope 1 until it stops.

Furthermore, because the stent 30 and the stent installation member 35 are preliminarily provided around the guide catheter 25 and pushing member 24, the stent delivery catheter 20 contributes to reducing the burden of placing the stent 30 during surgery and lessening the surgery time.

The stent used as one of the embodiments of the present invention can be as follows.

As shown in FIG. 7, a stent 60 comprises a main body 61 which is a tubular blocking part provided with a through-hole. The circumference of a distal end 61a of the main body 61 is tapered so as to reduce the outer diameter of the distal end 61a of the main body 61 toward its distal end. On the more proximal side than the chamfering part of the distal end 61a of the main body 61, a lateral hole 62 is provided which has an opening on the lateral face of the main body 61 which connects with the through-hole of the main body 61 in a perpendicular direction. On the more proximal side than the lateral hole 62, a flap 63 is provided. The flap 63 is made so that a part of the main body 61 is peeled from the proximal end to the distal end of the stent 60 by lifting the distal end of the flap and the width of the flap 63 is reduced toward its distal end from the proximal end which is connected to the main body 61, and the flap 63 is made so as to be able to change shape by a force applied from outside. On the proximal end of the main body 61, a flap 64 is provided and its structure is the same as that of the flap 63 except that the distal end of the flap 64 is lifted toward the distal end of the main body 61.

The stent 60 has a longer distance from its distal end and to the flap 63 than the conventional stents and has a shorter distance from its proximal end to the flap 64 than the conventional stents. By keeping the distance on the distal end of the main body longer, the interference between the surface of the lumens like the bile duct W3 or the like and the distal end of the stent 60 is minimized. And by keeping the distance on the proximal end of the main stent body shorter, disruption of the flow of food or the like inside of the duodenum W1 is reduced because it keeps the amount of projection of the proximal end of the stent 60 out to the duodenum W1 small when the proximal end of the stent 60 is disposed inside of the duodenum W1 for example. Considering the insertion of the stent 60 into the bile duct W3 as above for example, preferably, the distance between the distal end 61a of the stent 60 to the flap 63 is 20 mm and the distance between the proximal end of the stent 60 to the flap 64 is 12 mm.

Because the distal end 61a of the stent 60 is tapered, it is possible to smoothly insert it into the duodenal papilla W2 or the like thereby reducing discomfort to the patient. Because the distal side of the stent 60 which is inserted into the bile duct W3 or the like is made longer than the conventional stents, damage or the like to the mucous membrane on the tube wall is reduced because the distal end 60a of the stent 60 does not easily become stuck in the wall surface. Furthermore, because the proximal end of the stent 60 is shorter, the flow of food or the like is kept smoother and an uncomfortable feeling in the patient is reduced. As with the previous embodiment of the stent, it is preferable to provide the stent 60 around the guide catheter 25 preliminarily. As a result, it is possible to avoid setting the direction of the stent 60 reversely which has a tapered distal end 61a or has a different length on the distal side 60a and the proximal side.

Furthermore, this embodiment of the present invention may be placed in the following case.

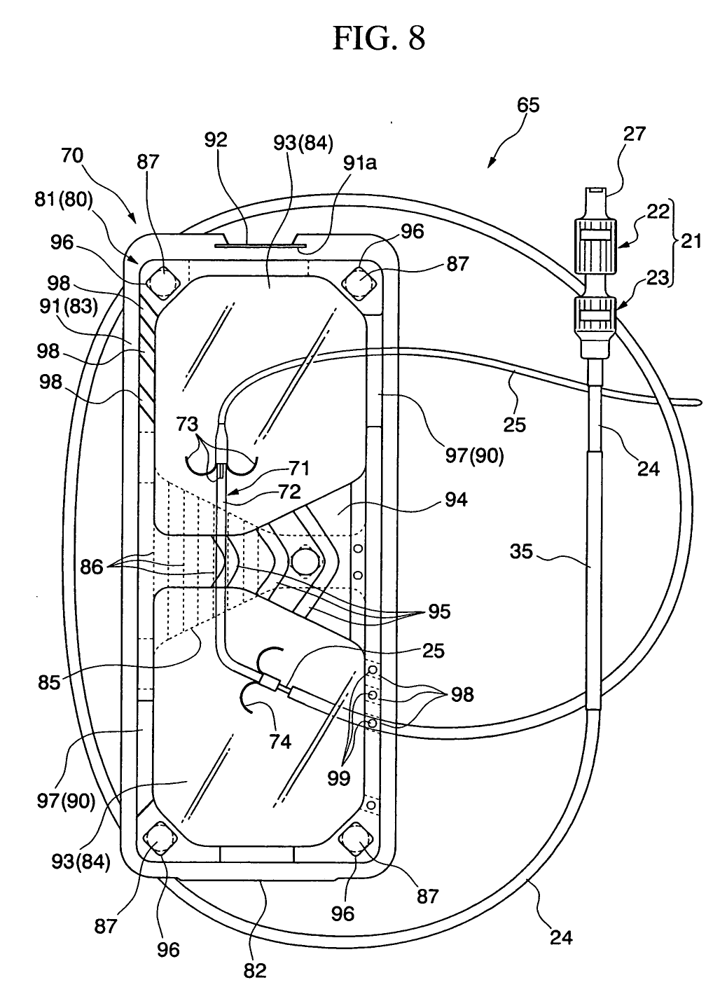

As shown in FIG. 8, a stent delivery catheter 65 is provided with a protective case 70. In this embodiment of the present invention, a stent 71 is provided around the guide catheter 25. The stent 71 comprises a bent main body 72 and four flaps 73 which are provided around the distal end of the main body 72 at equal intervals in the circumferential direction. Each of the flaps 73 is curved so that it opens toward the proximal end of the stent. In a similar way, on the proximal side, four flaps 74 are provided at equal intervals in the circumferential direction and each of the flaps 74 is curved so that it opens toward the distal end of the stent. This kind of stent 71 is used in a case, for example, to promote the expulsion of bile in the stenotic area when a stenosis developes in a bile duct.

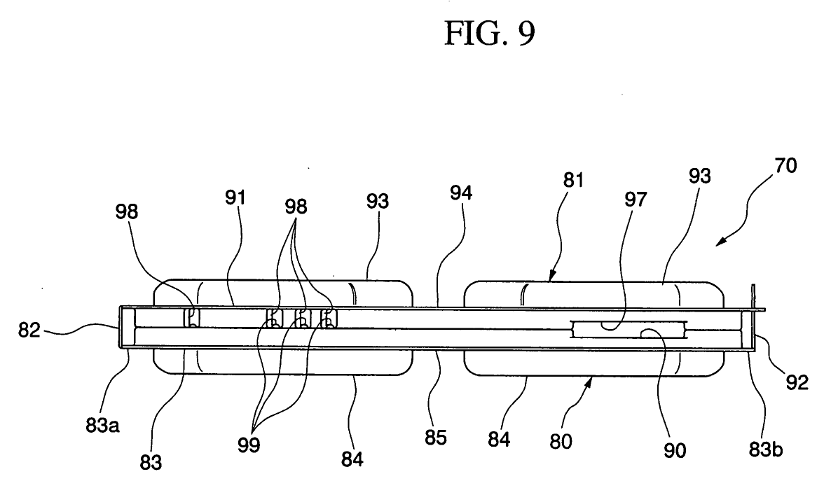

As shown in FIGS. 8 and 9, the protective case 70 is constructed so that a basement 80 and a lid 81 are connected via a connection part 82 and when overlapping the basement 80 with the lid 81 by bending the connection part 82, it is possible to house the stent 71, a part of the guide catheter 25 and a part of the pushing member 24 in the space created thereinside.

As shown in FIG. 9, the basement 80 comprises a housing part 84, except a flange 83 which is the surroundings of the basement 80, and a rib 85 which keeps the strength provided on substantially the center in the longitudinal direction on the basement 80. As shown in FIG. 8, the rib 85 is provided with a plurality of grooves 86 along the longitudinal direction of the rib 85 which is also the direction of the narrow side of the basement 80. These grooves 86 are made to removably hold the straight-conduit portion of the stent 71 and the shapes of the grooves are adjusted to the diameter or the shape of the stent 71. The flange 83 is provided with a plurality of convexes 87 which fit together with the lid 81 on the four corners.

On one lateral side of the longitudinal direction of the basement 80, a slit 90 is provided through which the guide catheter 25 passes. The width of the slit 90 is much bigger than the outer diameter of the guide catheter 25. On the flange 83 of the basement 80, the connection part 82 is provided on one fringe 83a which is in the direction of narrow side of the basement 80 and the basement 80 and the lid 81 is formed integrally via the connection part 82. On another fringe 83b, a tongue 92 is provided on the extension of the fringe 83b which is to be held with a flange 91 of the lid 81.

The lid 81 is comprised of a housing part 93, except for the flange 91 which is in the surroundings of the basement 81, and a rib 94 on substantially the center in the longitudinal direction on the lid 81. The rib 94 is provided with a plurality of grooves 95 which are made to removably hold the straight-conduit portion of the stent 71 along the longitudinal direction of the rib 94. These grooves 95 are made in a bent shape to hold the bent portion of the stent 71. The flange 91, which is overlapped with the flange 83 of the basement 80, is provided with a plurality of concaves 96 on the four corners which fit together with the convexes 87 of the flange 83.

Furthermore, on one lateral side of the longitudinal direction of the lid 81, a slit 97 is provided through which the guide catheter 25 passes. The slit 97 is formed on a position which overlaps with the slit 90 and formed in the same shape as the slit 90 and the slit 97 creates a space with the slit 90 through which the guide catheter 25 is inserted. A plurality of slits 98 are provided in a symmetrical position against the slit 90 across the rib 94 on one lateral side of the lid 81. Each slit 98, which is to be inserted through by the correctly positioned pushing member 24, has a width substantially the same as the outer diameter of the pushing member 24 and a plurality of the slits 98 are provided on the lid 81 along its longitudinal direction. Each slit 98 is slanted off in the longitudinal direction of the lid 81. In order to prevent the pushing member 24 from deviating, a plurality of swells 99 is provided at a location symmetrical against each of the slits 98. As shown in FIG. 8, on the flange 91 of the lid 81, an engagement 91a is provided to hold the tongue 92.

This stent delivery catheter 65 which is covered by the protective case 70 is stored in a sterile pack. The height of the housing parts 84 or 93 of the protective case 70 is much higher than the projected amount of the flap 73 or 74 and the housing part 84 or 93 has sufficient resistance against an external force. Therefore, in this state, the flap 73 or 74 of the stent 71 is kept open without being interfered with by the wall of the protective case 70 and their deformation is restrained. And the deviation of the stent 71 is also restrained because it is held by the groove 86. When the stent delivery catheter 20 is used, it is taken out of the sterile pack and then the protective case 70 is opened to remove the pushing member 24 out of the slit 98 and pick up the stent 71 out of the groove 86. Subsequent method of usage is the same as that of the first embodiment of the present invention.

As described above, because it is possible to store and use the stent delivery catheter 65 as one integrated unit, handling thereof becomes easy. And because the stent 71 is held by the protective case 70, deformation or the like of the flap 73 or 74 more than necessary can be avoided.

Furthermore, by providing the slit 98 on the protective case 70, it is possible to hold the pushing member 24 in a fixed state and this protects the stent 71 from being subjected to unexpected stresses. Because the guide catheter 25 passes through the wide slit 90 or 97, even when the stent 71 is held by any of the grooves 86, unexpected forces are not applied to the guide catheter 25.

When the stent 71 is held by the groove 95 on the lid 81, the guide catheter 25 and the pushing member 24 are inserted through the slits 90, 97 and 98 provided on the opposite side of the stent delivery catheter 65 shown in FIG. 8. And the stent installation member 35 may be held by either of the groove 86 or the groove 95.

Next, the second embodiment of the present invention is explained by referring to the figures. The same symbols are provided for the structural members common to the first embodiment of the present invention. Explanation is omitted of the structural members already explained above.

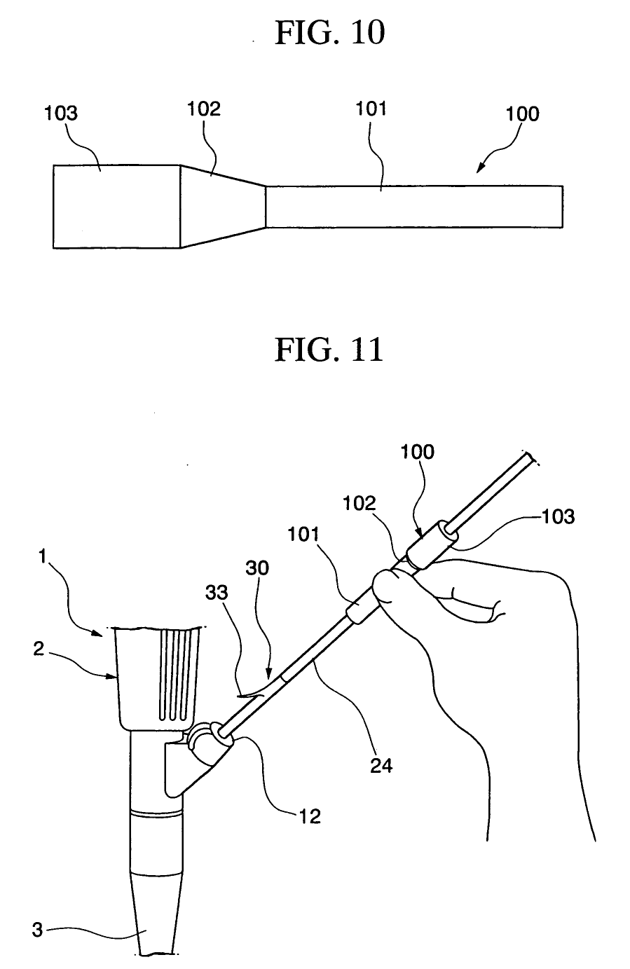

As shown in FIG. 10, a stent installation member 100 comprises a tubular main body 101 which functions as a blocking part and a tapered transition part 102 which functions as a regulative part on the proximal side of the main body 101. The outer diameter of a proximal end part 103 of the stent installation member 100 is larger than that of the main body 101 due to the transition part 102. The main body 101, transition part 102 and the proximal end part 103 of the stent installation member 100 are integrally formed and they are shaped in a hollow tubular shape. The main body 101 is the blocking part provided with an outer diameter so that it can be inserted into the first conduit 9 and further into the bifurcation 7 from the insertion slot 12. The inner diameter of the main body 101 is made so that the stent 30 can be inserted in a state such that the flaps 32 and 33 are closed. The length of the main body 101 is longer than the route from the insertion slot 12 to the bifurcation 8 and is shorter than the pushing member 24 and is preferably, for example, 60 mm. And the proximal end part 103 is provided with an outer diameter larger than the insertion slot 12 and is provided with an inner diameter so that the stent 30 can be inserted. The transition part 102 is made, for example, by thermoforming a tube, by covering a heat-shrinkable tube so as to have a step, or by connecting different tubes with different outer diameters.

The function of the stent installation member 100 will be explained.

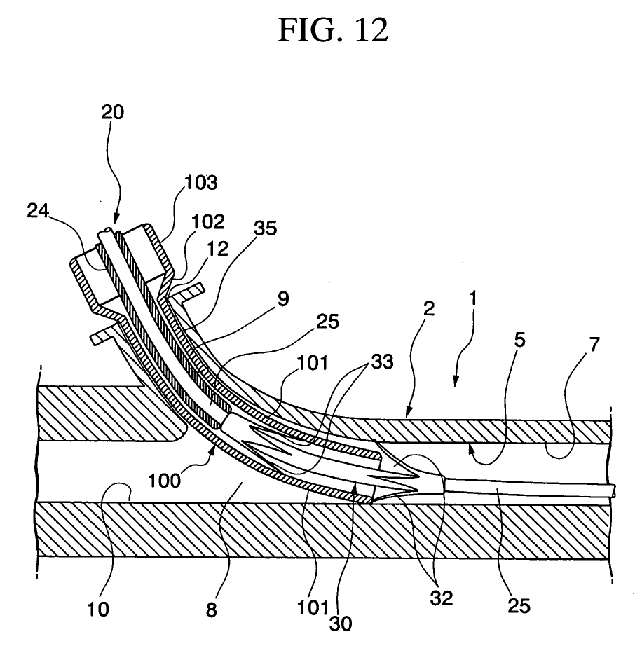

As shown in FIG. 11, the distal end of the guide catheter 25 and the distal end of the stent 30 are inserted into the treatment instrument insertion channel 5 from the insertion slot 12 of the endoscope 1. Before the flap 33 on the proximal side of the stent 30 is inserted from the insertion slot 12, the stent installation member 100 is inserted from the insertion slot 12. As shown in FIG. 12, the stent installation member 100 is inserted until the transition part 102 reaches the insertion slot 12. At this time, the distal end of the main body 101 reaches into the main conduit 7 passing through the first conduit 9 and the bifurcation 8. In this state, when the stent 30 is further inserted, the flap 33 on the proximal side of the stent 30 is covered inside of the stent installation member 100 and the stent reaches the main conduit 7 through the bifurcation 8.

In this manner, when the stent installation member 100 is used, by the transition part 102 being caught by the insertion slot 12, the insertion amount of the stent installation member 100 is regulated so as to be most suitable for the installation of the stent 30, accordingly, the stent installation member 100 is prevented from being inserted more than necessary into the endoscope 1. Other functions and effects are the same as those of the first embodiment of the present invention as described above.

In this embodiment of the present invention, the following stent installation member can be also used.

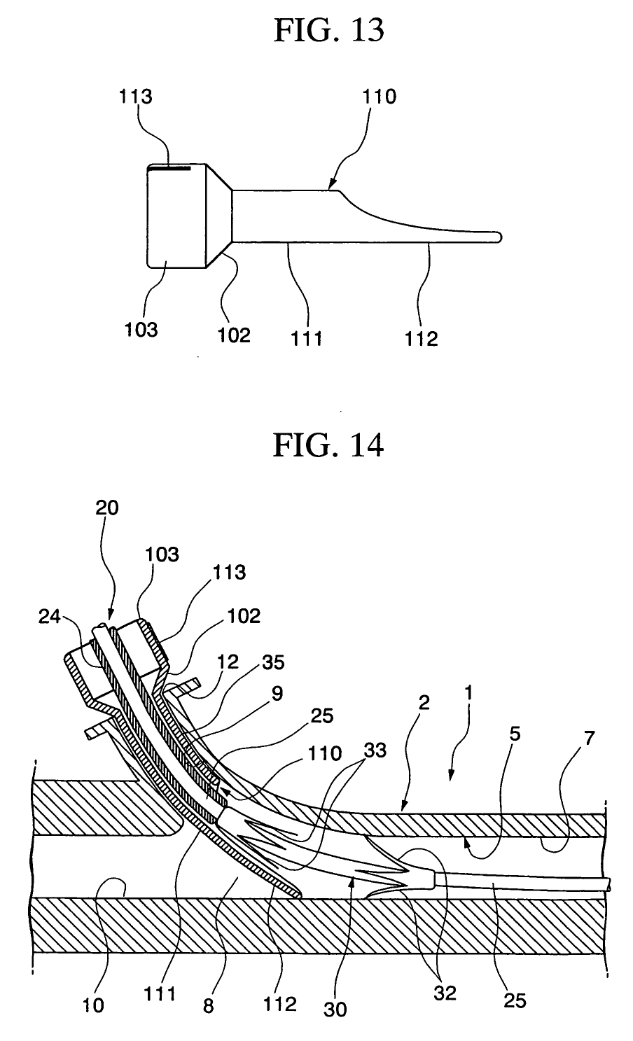

As shown in FIG. 13, a stent installation member 110 comprises a tubular main body 111 and a proximal part 103 provided on the proximal end of the main body 111 which has a larger diameter by being connected to the transition part 102. On the extension of the distal end of the main body 11, a blocking member 112 is provided. The blocking member 112 comprises a part of the circumference of the main body 111 extending along the axis of the main body. The inner diameter of the main body 111 is made so that it can house the stent 30 in a state such that the flaps 32 and 33 are closed, and the outer diameter of the main body 111 is smaller than the diameter of the first conduit 9. The length of the main body 111 is shorter than the total length of the first conduit 9, however, as a whole stent installation member 110, it is long enough to reach the main conduit 7 through the bifurcation 8 from the insertion slot 12. The length of the main body 111 is preferably, for example, approximately 30 mm and the length of the blocking part 112 in this case is approximately 30 mm.

A marker 113 is provided on the proximal part 103 of the stent installation member 110 so that the position where the blocking part 112 is extended can be identified in the circumferential direction. The marker 113 is provided at a position shifted by 180 degrees in the circumferential direction from the extended part of the blocking part 112. In other words, on the stent installation member 110 a part of which is slit in the circumferential direction, a marker 113 is provided on a corresponding part of the stent installation member 110 which is slit.

The stent installation member 110 is inserted into the insertion slot 12 when the stent 30 is inserted. The stent installation member 110 is inserted so that the marker 113 of the proximal part 103 is directed to the insertion part 3 of the endoscope, that is, to the downside in FIG. 1, and the stent installation member is inserted until the transition 102 contacts the insertion slot 12. As shown in FIG. 14, when the stent installation member 110 is inserted in this direction, it is inserted so that the blocking part 112 heads upward at the bifurcation 8 so as to block the opening communicating with the second conduit 10. When the stent 30 is sent toward the main conduit 7, in this state, one piece of the flap 33 which is provided on the proximal side of the stent 30 and facing the opening communicating with the second conduit 10 maintains contact with the blocking part 112 and is guided into the main conduit 7. As a result, the flap 33 does not open in the bifurcation 8 and is guided into the main conduit 7.

In the case that the stent installation member 110 is used, because the flap 33 does not open in the bifurcation 8, the flap 33 is prevented from being bent or the like. And the stent installation member 110 is made so that the blocking part 112 is the extended part of the main body 111, the stent installation member 110 is easy to transform along the conduit and therefore increases workability. By providing the transition part 102, it is possible to regulate the insertion amount of the stent installation member 110 and therefore it is possible to insert the stent 30 without fail. In this case, by providing the marker 113 on the proximal end part 103, it is easier to check the position of the blocking part 112 visually and therefore it is possible to position the blocking part 112 at the opening communicating with the second conduit 10 in the bifurcation 8 without fail.

Next, a third embodiment of the present invention is explained referring to the figures. The same symbols are provided for the structural members which are common to the above-described embodiment of the present invention. Explanation is omitted of the structural members already explained above.

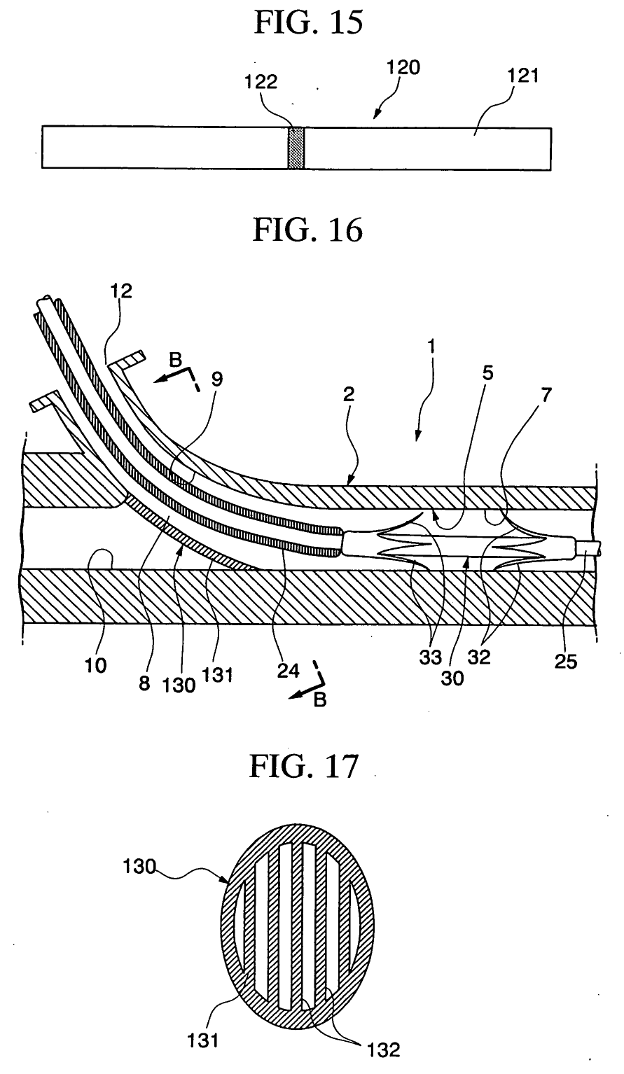

As shown in FIG. 15, a stent installation member 120 is provided with a main body 121 which is a tubular blocking part and on a circumferential surface from the distal end to the proximal end of the stent installation member a marker 122, which is an annular identification member, is provided. The marker 122 is provided at a position which corresponds to the length from the insertion slot 12 to the main conduit 7 through the bifurcation 8 and it is provided approximately 60 mm from the distal end of the stent installation member 120 for example. The inner diameter, outer diameter and the length of the stent installation member 120 are the same as those of the first embodiment of the present invention.

A function of the stent installation member 120 will be explained.

The installation member 120 is inserted into the opening 12 until the marker 122 reaches the opening 12. At this time, the distal end of the main body 121 reaches the main conduit 7 through the bifurcation 8. And when the stent 30 is guided into the main conduit 7 through the inside of the stent installation member 120, the stent 30 is installed with its flap 33 closed.

According to the stent installation member 120, by visually checking the marker 122, it is possible to identify the inserted amount of the stent installation member 120, and the stent installation member 120 can be prevented from being inserted too much into the endoscope 1. Other functions and effects of the stent installation member 120 are the same as those of the first embodiment of the present invention.

An embodiment of the marker 122 may be provided by painting the main body 121 in a different color. In particular, the distal side and proximal side from the position corresponding to the marker 122 is identified in different colors.

Next, a fourth embodiment of the present invention is explained referring to the figures. The same symbols are provided for the structural members which are common to the above-described embodiment of the present invention. Explanation is omitted of the structural members already explained above.

As shown in FIG. 16, the treatment instrument insertion channel 5 which is a conduit is provided with a regulative member 130 in the bifurcation 8. The regulative member 130 comprises a substantially circular plate-shaped main body 131 connecting with the first conduit 9 and the main body 131 is fixed so that it crosses the bifurcation 8 at a slant toward the main conduit 7 to block a path communicating with the second conduit 10. Furthermore, as shown in FIG. 17, a plurality of slits 132 is provided in parallel on the main body 131. These slits 132 provide communication between the bifurcation 8 and the second conduit 10 and the slit 132 shapes the main body 131 as a grid except for its circumferential portion.

When the stent 30 is inserted by using the treatment instrument insertion channel 5, the guide catheter 25 on which the stent is provided thereon is inserted from the insertion slot 12. The stent 30 is inserted into the main conduit 7 from the first conduit 9 through the bifurcation 8 and because the movement of the stent 30 from the bifurcation 8 into the second conduit 10 is regulated by the regulative member 130, the stent 30 is guided into the main conduit 7 so as to be guided by the main body 131 of the regulative member 130. Because the inner diameter of the bifurcation 8, where the first conduit 9 proceeds to the second conduit 10, is kept substantially the same as that of the inner diameter of the first conduit 9, the stent 30 is inserted into the main conduit 7 without the flap 33 of the stent 30 open. On the other hand, when normal saline or the like is supplied from the second conduit, it passes through the slit 132, the bifurcation 8 and the main conduit 7 and is supplied into the human body.

According to the embodiment of the present invention, because the regulative member 130 is provided in the treatment instrument insertion channel 5, it is possible to reduce the inner diameter of the conduit when the stent 30 is inserted and because of this the flap 32 of the stent 30 does not open inside the treatment instrument insertion channel 5 and the flap 32 is prevented from being bent or the like. And there is no need to add special structures on the stent delivery catheter side.

The invention is not limited to the above mentioned embodiments but can be applied broadly.

For example, the stent installation member may be comprised of only a plate-like member which is extended from the blocking part 112. By using this kind of stent installation member, the same function and effect as those of the main body 111 and the blocking part 112 of the stent installation member 110 can be obtained. In this state, the marker 122 may be provided on the proximal end of the blocking part 112 to be able to check visually the insertion amount of the blocking part 112.

The first treatment instrument is not limited to the stent delivery catheter but it can be applied to a guide wire provided with a bending at the tip of a sheath already, a papilotomy knife and angiographic catheter. In the case of using this kind of treatment instrument, the insertion of the stent from the bifurcation 8 into the second conduit 10 is prevented and because of this the stent is prevented from becoming stuck inside of the treatment instrument insertion channel 5, making quick insertion and pulling out of the stent possible.

Further, according to another embodiment of the present invention, the stent delivery catheter is provided with a protective case which houses the stent delivery catheter in a state such that the stent is pulled over around the guide member and the flaps of the stent are open.

Because the stent delivery catheter is provided with the protective case, the flaps of the stent are prevented from being bent or the like while the stent delivery catheter is stored.

The insertion method according to the present invention is comprised of the stent installation member insertion process and the stent insertion process. The stent installation member insertion process is utilized when the stent, provided with flaps on the distal end and proximal end of the stent to be placed in human body, is inserted from the insertion slot provided in the divaricated conduit divaricated at the bifurcation from the main conduit of the endoscope which has an opening on its distal end. The stent installation member insertion process is to insert the stent installation member from the insertion slot of the divaricated conduit and to make the distal end of the stent installation member pass over the bifurcation and reach the main conduit after inserting the flap of the stent provided on its proximal end inside the stent installation member, which is longer than the route from the insertion slot to the bifurcation of the main conduit, and the stent can be inserted. The stent insertion process is to make at least the flap of the stent provided on its proximal end pass over the bifurcation through the stent installation member after the stent installation member insertion process. According to this stent installation method, because the stent installation member insertion process is provided, the flap is prevented from opening in the bifurcation by reducing the apparent inner diameter of the conduit in the bifurcation when the stent passes over the bifurcation. As a result, unnecessary forces are not applied to the flap when the stent proceeds into the main conduit from the bifurcation.

According to the treatment instrument installation member of the present invention, the diameter of the first conduit through which the first treatment instrument is inserted decreases because if the main body is inserted into the first conduit, the distal end of the main body blocks a part of the opening at the connection part.

According to this installation member of the present invention, a conduit which has a smaller inner diameter than that of the connection part is created by the main body through which the first treatment instrument is inserted.

According to this installation member of the present invention, an insertion into the correct location can be achieved by the installation amount being regulated by the regulative part.

According to the stent delivery catheter of the present invention, the insertion of the stent can be performed with a part of the opening of the connection part blocked by the stent installation member, and therefore the bending of the flap of the stent, which is caused by the opening of the flap, is prevented when the stent is passing through the connection part.

According to the stent delivery catheter of the present invention, flaps of the stent are prevented from being bent during storage by being provided with a protection case.

According to the endoscope treatment system of the present invention, the stent can be inserted in the correct condition because the flap of the stent is prevented from being bent at the connection part when the stent is inserted into the human body.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A treatment instrument installation member which is utilized for the insertion of a first treatment instrument from a first conduit of a conduit system comprising:
the first conduit provided with an inner diameter through which the first treatment instrument can be inserted,
a second conduit provided with an inner diameter through which a second treatment instrument can be inserted,
a main conduit provided with an inner diameter through which the first treatment instrument and the second treatment instrument can be inserted, and
a connection part which connects the first conduit, the second conduit and the main conduit,
wherein the treatment instrument installation member further comprising:
a main body provided with an outer diameter which can be inserted into the first conduit, and
a distal end part located on the proximal side of the main body, and provided with a blocking part which blocks at least a part of an opening communicating with the second conduit at the connection part.

2. The treatment instrument installation member according to claim 1, wherein
the main body is a tubular member provided with an outer diameter smaller than the inner diameter of the first conduit and the connection part, and is also provided with an inner diameter larger than the outer diameter of the first treatment instrument, and
the main body is exposed from the conduit system at a first insertion slot of the treatment instrument, which is located on an operation part side of the conduit system, while the distal end part blocking at least a part of the opening communicating with the second conduit at the connection part.

3. The treatment instrument installation member according to claim 1 or 2, wherein
the main body is provided with a regulative part on the proximal side to regulate the insertion amount into the first conduit.

4. The treatment instrument installation member according to claim 1 or 2, wherein
the blocking part comprises a part of the circumference of the main body extending along the axis of the main body, and
a marker, which is a lid part, formed along the axis of the treatment instrument installation member, is provided on the circumference of a proximal part of the treatment instrument installation member at a position shifted by 180 degrees in the circumferential direction from the blocking part to locate the blocking part in a position where at least a part of the opening communicating with the second conduit is reliably blocked at the connection part.

5. The treatment instrument installation member according to claim 3, wherein
the blocking part comprises a part of the circumference of the main body extending along the axis of the main body, and
a marker, which is a lid part, formed along the axis of the treatment instrument installation member, is provided on the circumference of a proximal part of the treatment instrument installation member at a position shifted by 180 degrees in the circumferential direction from the blocking part to locate the blocking part in a position where at least a part of the opening communicating with the second conduit is reliably blocked at the connection part.

6. A stent delivery catheter utilized with an endoscope provided with
a first conduit provided with an inner diameter through which a first treatment instrument can be inserted,
a second conduit provided with an inner diameter through which a second treatment instrument can be inserted,
a main conduit provided with an inner diameter through which the first treatment instrument and the second treatment instrument can be inserted, and
a connection part which connects a first conduit, a second conduit, and a main conduit,
wherein the stent delivery catheter comprising:
a guide member inserted to the first conduit and the main conduit from the first insertion slot of the treatment instrument which is located on an operation part of the endoscope,
a stent movably provided on the guide member,
a pushing member movably provided forward and backward on the guide member to push out the stent to place into position, and
a stent installation member, which is a treatment instrument installation member according to claim 1, movably provided forward and backward on the pushing member and on the stent.

7. The stent delivery catheter according to claim 6, wherein
the stent is provided with flaps which regulates movement of the stent, and
the stent is further provided with a protective case, wherein
the protective case holds the stent in a state such that the stent is provided on the guide member, and
the protective case covers the stent in a state such that the flaps are open.

8. An endoscope treatment system provided with the endoscope and the stent delivery catheter according to claim 6.

9. A stent delivery catheter according to claim 7, wherein
the stent has flaps on the distal side of the stent and on the proximal side of the stent, and
the length between the proximal end of the stent and the flap on the proximal side of the stent is shorter than the length between the distal end of the stent and the flap on the distal side of the stent.

10. A conduit system comprising:
a first conduit provided with an inner diameter through which a first treatment instrument can be inserted,
a second conduit provided with an inner diameter through which a second treatment instrument can be inserted,
a main conduit provided with an inner diameter through which the first treatment instrument and the second treatment instrument can be inserted,
a connection part which connects the first conduit, the second conduit, and the main conduit, and
a regulative member located in the connection part, wherein
the regulative member restrains the insertion of the first treatment instrument which is extended from the first conduit into the second conduit, and
the regulative member enables the insertion of the second treatment instrument which is extended from the second conduit into the main conduit.

11. The conduit system according to claim 10, wherein
the regulative member has a substantially circular plate-shaped main body connecting with the first conduit, and the main body of the blocking member is fixed so that it crosses the bifurcation at a slant toward the main conduit.

12. The conduit system according to claim 11, wherein
a plurality of slits is provided in parallel on the substantially circular plate-shaped main body of the regulative member.

13. A stent installation method to install a stent provided with flaps on its distal end and proximal end to be placed in the human body from an insertion slot provided in a divaricated conduit divaricated at a bifurcation from a main conduit of an endoscope which has an opening on its distal end,
the method comprising:
a stent installation member insertion process to insert the stent installation member, through which the stent can be inserted, from the insertion slot of the divaricated conduit and to make the distal end of the stent installation member pass over the bifurcation from the divaricated conduit to reach the main conduit, after locating at least the flap of the stent provided on its proximal end in the stent installation member which is longer than the route from the insertion slot to the bifurcation of the main conduit, and
a stent insertion process to make at least the flap of the stent provided on its proximal end pass over the bifurcation by proceeding through the stent installation member after the stent installation member insertion process.
